(19) United States
(12) Patent Application Publication
(10) Pub. No.: US 2026/0174591 A1
(43) Pub. Date: Jun. 25, 2026

(54) Thermal Contact Pad Assembly

(71) Applicant: Becton, Dickinson and Company, Franklin Lakes, NJ (US)

(72) Inventors: Gabriel A. Johnston, Broomfield, CO (US); Madeline Stich, Thornton, CO (US)

(21) Appl. No.: 18/988,717

(22) Filed: Dec. 19, 2024

(51) Int. Cl. A61F 7/00; A61F 7/08

(52) U.S. Cl. CPC A61F 7/0085; A61F 2007/0054; A61F 2007/083

(57) ABSTRACT

A thermal contact pad assembly configured for use in conjunction with a targeted temperature management (TTM) system module which circulates TTM fluid through the thermal contact pad assembly. The thermal contact pad assembly includes a plurality of thermal contact pads fluidly coupled together which, during use, can be decoupled from each other. Fluid connectors between the pads include valves that open and close upon connection and disconnection of the fluid connectors. Each of the pads is covered by a fabric wrap and connection mechanisms of the fabric wraps enable selective attachment and detachment of the fabric wraps to and from each other. A method of using the thermal contact pad assembly includes decoupling one of the thermal contact pads from the remaining thermal contact pads and coupling a replacement thermal contact pad in place thereof while maintaining circulation of the TTM fluid through the remaining pads.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. A fluid delivery transports TTM fluid between TTM system module and the thermal pad. In some instances of TTM therapy, two or more thermal pads may be used. Maintaining the thermal contact pads in thermal contact with the patient can be difficult, especially during patient movement. In some instances of a TTM therapy, it may be advantageous to replace one or more of a plurality of thermal contact pads during the TTM therapy which can require temporarily suspending the TTM therapy during the replacement resulting in effectiveness decrease for the TTM therapy. In additional, replace one or more of a plurality of thermal contact pads can result in leaking or spilling the TTM fluid causing further interruption and/or added therapy cost. Disclosed herein are embodiments of systems, devices, and methods for securing the thermal pads to the patient and minimizing therapy interruption during replacement of a thermal pad.

### SUMMARY

Disclosed herein is a thermal contact pad assembly (pad assembly) configured for use in conjunction with a targeted temperature management (TTM) system. The pad assembly includes, according to some embodiments, a plurality of thermal contact pads (pads), where each pad includes a fluid compartment configured for circulation of a targeted TTM fluid (at a defined temperature) therein to define a thermal energy exchange between the TTM fluid and a patient, i.e., to warm or cool the patient. Each pad includes an inlet connector (i.e., one of a plurality of inlet connectors of the pad assembly in fluid communication with the fluid compartment), where the inlet connector is configured to couple with any one of a plurality of corresponding fluid delivery connectors, where each fluid delivery connector may be a component of one of the pads or a fluid delivery line (FDL) of the TTM system. Each pad also includes an outlet connector (i.e., one of a plurality of outlet connectors of the pad assembly) in fluid communication with the fluid compartment, where each outlet connector is configured to couple with any one of a plurality of corresponding fluid return connectors, where each fluid return connector may be a component of one of the pads or the FDL.

Each pad includes a fabric wrap that defines a covering for the fluid compartment of the pad. The fabric wrap includes an outer panel attached to a patient contact panel attached along a perimeter of the fabric wrap, such that the fluid compartment is (i) disposed between the outer panel attached and the patient contact panel, and (ii) enclosed by the fabric wrap. The fabric wrap includes one or more attachment mechanisms that are configured to attach the pad to another one of the pads.

In some embodiments, the one or more attachment mechanisms are configured for selective (i) attachment of the pad to the other one of the pads and (ii) detachment of the pad from the other one of the pads. In some embodiments, the pad assembly further includes one or more patient attachment mechanisms configured to secure the pad assembly to the patient. In some embodiments, the patient attachment mechanisms are configured to secure one portion of the pad to another portion of the pad.

In some embodiments, each inlet connector includes a valve disposed in line therewith, where the valve is configured to (i) transition from a closed state to an open state upon coupling (i.e., as a result of coupling) of the inlet connector with the corresponding fluid delivery connector, and (ii) transition from the open state to the closed state upon decoupling (i.e., as a result of decoupling) of the inlet connector from the corresponding fluid delivery connector. Similarly, each outlet connector includes a valve disposed in line therewith, where the valve is configured to (i) transition from a closed state to an open state upon coupling (i.e., as a result of coupling) of the outlet connector with the corresponding fluid return connector, and (ii) transition from the open state to the closed state upon decoupling (i.e., as a result of decoupling) of the outlet connector from the corresponding fluid return connector. Further, in some embodiments, each fluid delivery connector and fluid return includes a similar valve disposed in line therewith.

In some embodiments, each of the pads are configured for use individually in conjunction with the TTM system. In some embodiments, each of the pads are configured for coupling with the FDL, where the FDL includes at least one of the fluid delivery connectors and at least one of the fluid return connectors.

In some embodiments, one or more of the pads includes one or more of the corresponding fluid delivery connectors in fluid communication with the fluid compartment and one or more of the corresponding fluid return connectors in fluid communication with the fluid compartment.

In some embodiments, each of the corresponding fluid delivery connectors included by the one or more of the pads includes a fluid restrictor disposed in line therewith, where the fluid restrictor is configured maintain a desired circulation of the TTM fluid within the fluid compartment of the corresponding one or more of the pads.

In some embodiments, the plurality of pads includes a first pad, a second pad, and a third pad, where each of the first, second, and third pads defines a top side, a bottom side disposed opposite the top side, a left side, and a right side disposed opposite the left side. In some embodiments, when the pad assembly is disposed in an assembled state, the bottom side of the first pad is disposed adjacent the top side of the second pad and the top side of the third pad, and the right side of the second pad is disposed adjacent the left side of the third pad.

In some embodiments, the first pad is attached to the second pad and the third pad via the one or more attachment mechanisms.

In some embodiments, the inlet connector and the outlet connector of the second pad are coupled with a first delivery connector and a first return connector of the first pad, respectively. In some embodiments, the inlet connector and the outlet connector of the third pad are coupled with a second delivery connector and a second return connector of the first pad, respectively.

In some embodiments, the first pad is divided into a first sub-pad and a second sub-pad such that the fluid compartment of the first pad is divided into a first sub-fluid compartment and a second sub-fluid compartment. The first sub-pad includes the inlet connector and the outlet connector of the first pad which are in fluid communication with the first sub-fluid compartment. The second sub-pad includes one of the plurality of inlet connectors and one of the plurality of outlet connectors in fluid communication with the second sub-fluid compartment. The first and second sub-pads include first and second sub-fabric wraps defining a covering therefor, where each of the first and second sub-fabric wraps includes an individual outer panel and an individual patient contact panel that is attached to the individual outer panel along a perimeter of the corresponding sub-fabric wrap. Each of the first and second sub-fabric wraps includes a subset of the one or more attachment mechanisms, where the subset is configured to attach first sub-pad to the second sub-pad.

Also disclosed herein is a method of exchanging thermal energy with a patient that, according to some embodiments, includes fluidly coupling a thermal contact pad assembly to a system module of a targeted temperature management (TTM) system, where the thermal contact pad assembly includes a plurality of thermal contact pads (pads), and where each pad includes (i) a fluid compartment configured for circulation of a targeted TTM fluid therein to define a thermal energy exchange between the TTM fluid and the patient and (ii) a fabric wrap defining a covering for the fluid compartment. According to such embodiments, the fabric wrap includes (i) an outer panel, (ii) patient contact panel attached to the outer panel along a perimeter of the fabric wrap, and one or more attachment mechanisms configured to secure the pad to an adjacent one of the pads. The method further includes (i) applying the thermal contact pad assembly to the patient, (ii) securing the thermal contact pad assembly to the patient via one or more patient attachment mechanisms of the thermal contact pad assembly, and (iii) circulating a TTM fluid through the fluid compartments to establish the thermal energy transfer.

In some embodiments, the method further includes (i) decoupling one pad of the thermal pad assembly from remaining pads of the thermal pad assembly and (ii) coupling a replacement pad to the remaining pads in place of the one pad during circulation of the TTM fluid through the remaining pads.

In some embodiments of the method, decoupling the one pad from the remaining pads includes decoupling one or more attachment mechanisms of the fabric wrap of the one pad from one or more attachment mechanisms of the fabric wrap of at least one of the remaining pads, and coupling the replacement pad to the remaining pads includes coupling one or more attachment mechanisms of the fabric wrap of the replacement pad to the one or more attachment mechanisms of the fabric wrap of the at least one of the remaining pads.

In some embodiments, the method further includes maintaining circulation of the TTM fluid within the fluid compartments of the remaining pads during the decoupling of the one pad and the coupling of the replacement pad.

In some embodiments of the method, decoupling the one pad from the remaining pads includes (i) decoupling a delivery connector of the remaining pads from an inlet connector of the one pad, and (ii) decoupling a return connector of the remaining pads from an outlet connector of the one pad, and coupling the replacement pad in place of the first pad includes (i) coupling the delivery connector of the remaining pads to an inlet connector of the replacement pad and (ii) coupling the return connector of the remaining pads to an outlet connector of the replacement pad.

These and other features of the concepts provided herein will become more apparent to those of skill in the art in view of the accompanying drawings and following description, which describe particular embodiments of such concepts in greater detail.

### DRAWINGS

FIG. 1 illustrates a targeted temperature management system in use with a patient including a thermal contact pad assembly secured to the patient, according to some embodiments disclosed herein.

FIG. 2A is a first exploded front view of the thermal contact pad assembly of FIG. 1 illustrating some details of the thermal contact pad assembly, according to some embodiments disclosed herein.

FIG. 2B is a second exploded front view of the thermal contact pad assembly of FIG. 1 illustrating further details of the thermal contact pad assembly, according to some embodiments disclosed herein.

FIG. 2C is a front view of the thermal contact pad assembly in an assembled state, according to some embodiments disclosed herein.

FIG. 2D is an illustration of the thermal contact pad secured to the patient, according to some embodiments disclosed herein.

FIG. 3 is an exploded front view of a second embodiment of a first thermal pad of the thermal contact pad assembly, according to some embodiments disclosed herein.

FIG. 4 is a fluid schematic illustration of the thermal contact pad assembly, according to some embodiments disclosed herein.

FIG. 5 is a block diagram of a method of exchanging thermal energy with the patient utilizing the thermal contact pad assembly, according to some embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. Furthermore, reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment”, “in an embodiment”, or “in some embodiments”, as well any other variations, appearing in various places throughout the specification are not necessarily all referring to the same embodiment or embodiments.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, fluid, and thermal interaction. Two components may be coupled with each other even though they are not in direct contact with each other. For example, two components may be coupled with each other through an intermediate component.

“Proximal” is used to indicate a portion, section, piece, element, or the like of a medical device or system intended to be near or relatively nearer to a clinician when the medical device or system is used on a patient. For example, “proximal” is used to indicate a portion, section, piece, element, or the like of the TTM system near or relatively nearer to the clinician such as the control module while the clinician operates the control module with the one or more pads placed around the patient. A “proximal portion” or “proximal section” of the medical device or system includes a portion or section of the medical device or system intended to be near the clinician when the medical device is used on the patient. Likewise, a “proximal length” of the medical device or system includes a length of the medical device or system intended to be near the clinician when the medical device is used on the patient. A “proximal end” of the medical device or system is an end of the medical device or system intended to be near the clinician when the medical device or system is used on the patient. The proximal portion, the proximal section, or the proximal length of the medical device or system need not include the proximal end of the medical device or system. Indeed, the proximal portion, the proximal section, or the proximal length of the medical device or system can be short of the proximal end of the medical device or system. However, the proximal portion, the proximal section, or the proximal length of the medical device or system can include the proximal end of the medical device or system. Should context not suggest the proximal portion, the proximal section, or the proximal length of the medical device or system includes the proximal end of the medical device or system, or if it is deemed expedient in the following description, “proximal portion,” “proximal section,” or “proximal length” can be modified to indicate such a portion, section, or length includes an end portion, an end section, or an end length of the medical device or system for a “proximal end portion,” a “proximal end section,” or a “proximal end length” of the medical device or system, respectively.

“Distal” is used to indicate a portion, section, piece, element, or the like of a medical device or system intended to be near or relatively nearer a patient when the medical device or system is used on the patient. For example, “distal” is used to indicate a portion, section, piece, element, or the like of the TTM system near or relatively nearer to the patient such as the one-or-more pads around the patient while a clinician operates the control module. A “distal portion” or “distal section” of the medical device or system includes a portion or section of the medical device or system intended to be near, relatively nearer, or even in the patient when the medical device or system is used on the patient. Likewise, a “distal length” of the medical device or system includes a length of the medical device or system intended to be near, relatively nearer, or even in the patient when the medical device or system is used on the patient. A “distal end” of the medical device or system is an end of the medical device or system intended to be near, relatively nearer, or even in the patient when the medical device or system is used on the patient. The distal portion, the distal section, or the distal length of the medical device or system need not include the distal end of the medical device or system. Indeed, the distal portion, the distal section, or the distal length of the medical device or system can be short of the distal end of the medical device or system. However, the distal portion, the distal section, or the distal length of the medical device or system can include the distal end of the medical device or system. Should context not suggest the distal portion, the distal section, or the distal length of the medical device or system includes the distal end of the medical device or system, or if it is deemed expedient in the following description, “distal portion,” “distal section,” or “distal length” can be modified to indicate such a portion, section, or length includes an end portion, an end section, or an end length of the medical device or system for a “distal end portion,” a “distal end section,” or a “distal end length” of the medical device or system, respectively.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

TTM systems circulate a TTM fluid (e.g., water) through one or more thermal contact pads coupled with a patient to affect surface-to-surface thermal energy exchange with the patient. In general, TTM systems comprise a TTM system module (i.e., a fluid control module) coupled with at least one thermal contact pad via a fluid deliver line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. Another TTM system including a TTM fluid control module coupled with number of thermal contact pads is disclosed in U.S. Published Application No. 2022-0287874 titled “Methods, Systems and Apparatus for Direct Connection of Gel Pad Targeted Temperature Management Device” filed Mar. 3, 2022, which is also incorporated herein by reference in its entirety. A fluid delivery line generally includes at least two fluid conduits for transporting TTM fluid to and from the thermal pad. In some instances of TTM therapy, two or more thermal pads may be used. A fluid delivery line generally includes a proximal portion having two fluid conduits extending distally from the TTM system module to the manifold and a distal portion having two fluid conduits for each thermal pad extending distally from the manifold to the thermal pads.

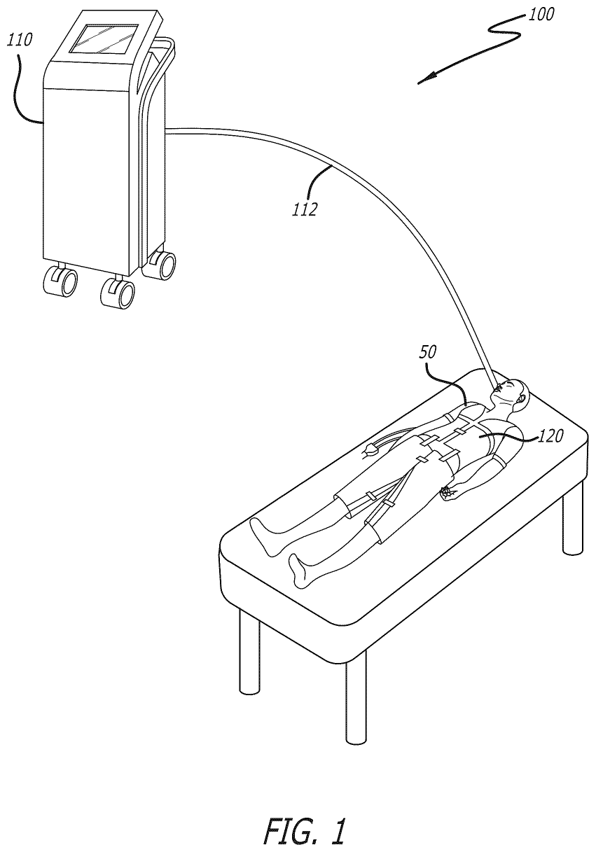

FIG. 1 illustrates an embodiment of a targeted temperature management (TTM) system 100 in use with a patient 50, according to some embodiments disclosed herein. FIG. 1 shows the TTM system 100 having a TTM module 110 fluidly coupled with a thermal contact pad assembly (pad assembly) 120 via a fluid deliver line (FDL) 112 such that a TTM fluid prepared by the TTM module 110 is circulated through the thermal contact pad assembly 120. The thermal contact pad assembly 120 is thermally coupled with the patient 50 such that a temperature difference between the TTM fluid circulating within the thermal contact pad assembly 120 and the patient 50 causes a thermal energy exchange with the patient 50 in accordance with a defined TTM therapy to cool or warm the patient 50.

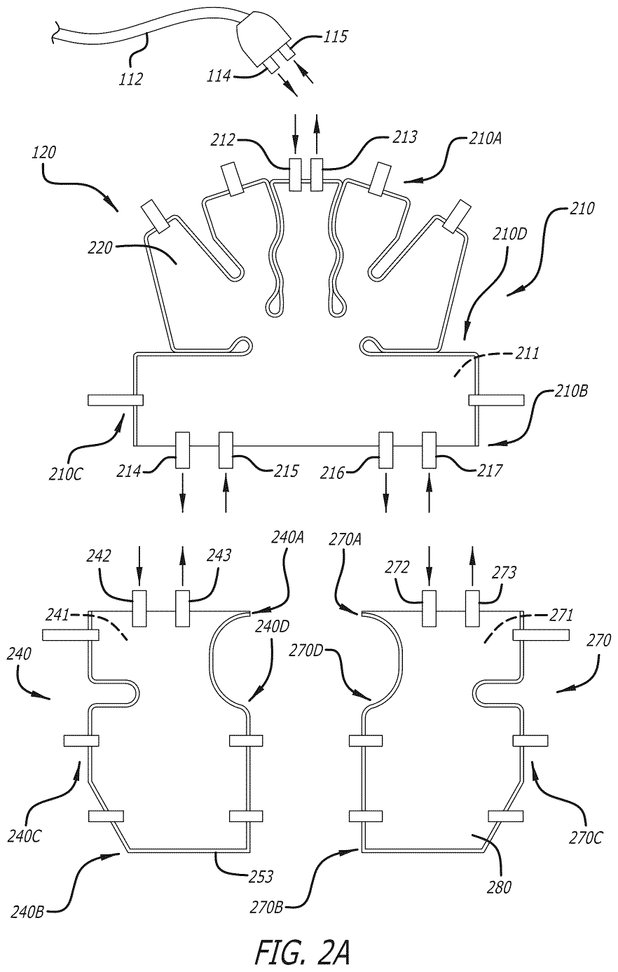

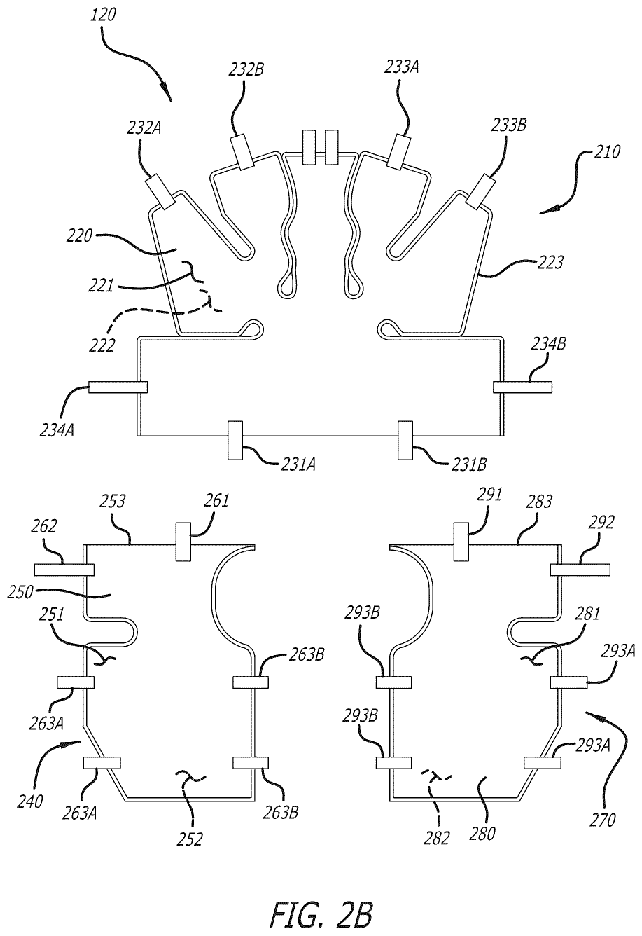

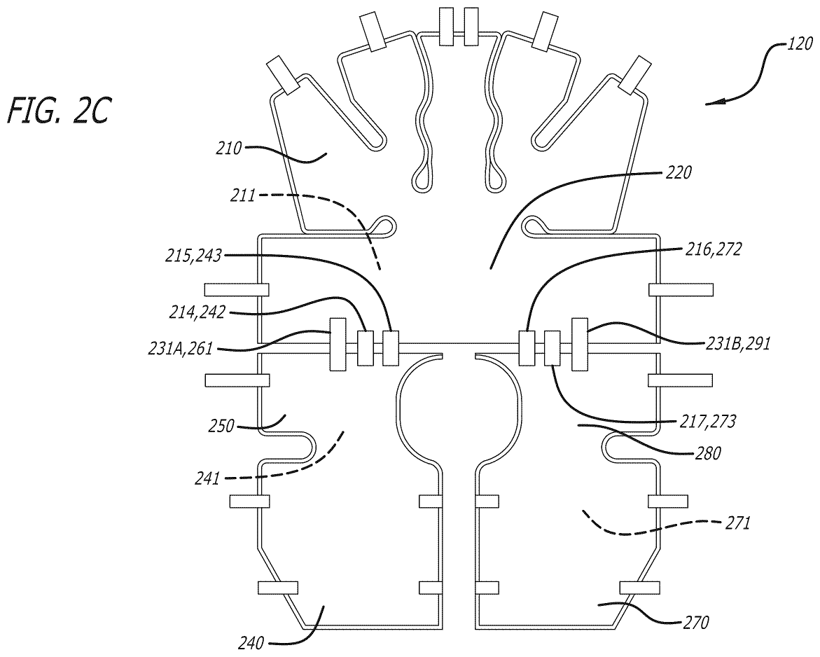

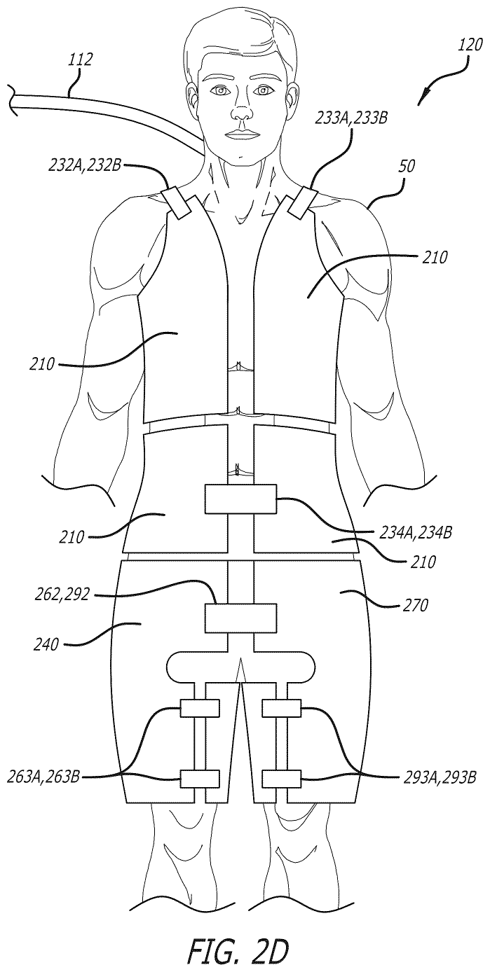

FIG. 2A is an exploded front view of the pad assembly 120 according to some embodiments disclosed herein. The pad assembly 120 generally includes a first pad 210, a second pad 240, and a third pad 270. Each of the second pad 240 and/or the third pad 270 may be selectively coupled with and decoupled from the first pad 210.

The first pad 210 may define a shape consistent with application to a torso portion of the patient extending substantially from a waist or hip to a shoulder area of the patient and extending around the patient. A front side of the first pad 210 may be applied to the patent such that a central portion of the first pad 210 is disposed across the back side (e.g., dorsum and lumbus) of the patient. Outer portions extending outward from the central portion may extend around the patient to the front side (e.g., thorax and abdomen) of the patient. For illustration purposes, the first pad 210 defines a first top side 210A, a first bottom side 210B, a first left side 210C, and a first right side 210D.

The second pad 240 may define a shape consistent with application to a right hip and thigh area of the patient. A front side of the second pad 240 may be applied to the right hip and thigh area such that an inner portion of the second pad 240 is disposed across the back side or right side of the right thigh and right buttock area. Outer portions of the second pad 240 extending outward from the inner portion of the second pad 240 may extend around the right thigh to the front side of the right thigh. For illustration purposes, the second pad 240 defines a second top side 240A, a second bottom side 240B, a second left side 240C, and a second right side 240D.

Similarly, the third pad 270 may define a shape consistent with application to a left hip and left thigh area of the patient. A front side of the third pad 270 may be applied to the left hip and thigh area such that an inner portion of the third pad 270 is disposed across the back side or left side of the left thigh and left buttock area. Outer portions of the third pad 27-extending outward from the inner portion of the third pad 270 may extend around the left thigh to the front side of the left thigh. For illustration purposes, the third pad 270 defines a third top side 270A, a third bottom side 270B, a third left side 270C, and a third right side 270D.

The first pad 210 include a first fluid compartment 211 that extends across a substantial entirety of an area (frontal area as shown) of the first pad 210. The first fluid compartment 211 includes a first inlet connector 212 and a first outlet connector 213. The TTM fluid enters the first fluid compartment 211 via the first inlet connector 212 and exits the first fluid compartment 211 via the first outlet connector 213. The first inlet connector 212 is configured to couple with a first delivery connector 114 of the FDL 112 and the first outlet connector 213 is configured to couple with a first return connector 115 of the FDL 112. As such, during operation TTM fluid prepared by the TTM system module 110 is (i) delivered to the first fluid compartment 211 via the first delivery connector 114 coupled with the first inlet connector 212, (ii) circulated through the first fluid compartment 211, and (iii) retuned to the TTM system module 110 via the first outlet connector 213 coupled with the first return connector 115.

The second pad 240 includes a second fluid compartment 241 having a second inlet connector 242 and a second outlet connector 243 such that the TTM fluid enters the second fluid compartment 241 via the second inlet connector 242 and exits the second fluid compartment 241 via the second outlet connector 243. Similarly, the third pad 270 includes a third fluid compartment 271 having a third inlet connector 272 and a third outlet connector 273 such that the TTM fluid enters the third fluid compartment 271 via the third inlet connector 272 and exits the third fluid compartment 271 via the third outlet connector 273.

The first fluid compartment 211 further includes a second delivery connector 214, a second return connector 215, a third delivery connector 216, and a third return connector 217 which are all in fluid communication with the first fluid compartment 211. When the second pad 240 is coupled with the first pad 210, the second delivery connector 214 is coupled with the second inlet connector 242 and the second return connector 215 is coupled with the second outlet connector 243. As such, a portion of the TTM fluid circulating within or passing through the first fluid compartment 211 is diverted to the second fluid compartment 241 for circulation within the second fluid compartment 241. Similarly, when the third pad 270 is coupled with the first pad 210, the third delivery connector 216 is coupled with the third inlet connector 272 and the third return connector 217 is coupled with the third outlet connector 273. As such, a portion of the TTM fluid circulating within or passing through the first fluid compartment 211 is diverted to the third fluid compartment 271 for circulation within the third fluid compartment 271.

In some embodiments, the second pad 240 may be configured to couple directly with the FDL 112 so that the second pad 240 may be utilized for thermal energy exchange singularly. As such, the second inlet connector 242 may be configured to couple with the first delivery connector 114 and the second outlet connector 243 may be configured to couple with the first return connector 115. Further in some embodiments, the third pad 270 may be configured to couple directly with the FDL 112 so that the third pad 270 may be utilized for thermal energy exchange singularly. As such, the third inlet connector 272 may be configured to couple with the first delivery connector 114 and the third outlet connector 273 may be configured to couple with the first return connector 115.

FIG. 2B is another exploded front view of the pad assembly 120, illustrating further details of the pad assembly 120, according to some embodiments disclosed herein. Each of the first, second, and third pads 210, 240, 270 includes a fabric wrap defining a covering for the first, second, and third fluid compartments 211, 241, 271, respectively. The first pad 210 includes a first fabric wrap 220 having a first patient contact panel 221 extending across the front side of the first pad 210 (i.e., the side facing out of the page), and a first outer panel 222 extending across a back side of the first pad 210 (i.e., the side facing into page), where the first patient contact panel 221 and the first outer panel 222 are attached together along a first perimeter seam 223, and where the first fluid compartment 211 is disposed between the first patient contact panel 221 and the first outer panel 222. Similarly, the second pad 240 includes a second fabric wrap 250 having a second patient contact panel 251 extending across the front side and second outer panel 252 extending across the back side, where the second patient contact panel 251 and the second outer panel 252 are attached together along a second perimeter seam 253, and where the second fluid compartment 241 is disposed between the second patient contact panel 251 and the second outer panel 252. The third pad 270 includes a third fabric wrap 280 having a third patient contact panel 281 extending across the front side and a third outer panel 282 extending across the back side, where the third patient contact panel 281 and the third outer panel 282 are attached together along a third perimeter seam 283, and where the third fluid compartment 271 is disposed between the third patient contact panel 281 and the third outer panel 282. The fabric wrap may be formed of any suitable woven or non-woven fabric material, such as a cotton, or synthetic material, for example.

In the illustration embodiment, each of the first, second, and third perimeter seams 223, 253, 283 may extend substantially around an entirety the respective pad. In other embodiments, one or more of the perimeter seams may include one or more segments where the patient contact pad is not attached to the outer pad. In some embodiments, each of the inlet, outlet, delivery and return connectors may pass through an open segment of the respective perimeter seam. Each perimeter seam may include any suitable attachment mechanism, such as sewing, heat bonding, adhesive bonding, and the like.

Each of the first, second, and third fabric wraps 220, 250, 280 may include essentially the same shape as the first, second, and third fluid compartments 211, 241, 271, respectively. Said another way, each of the first, second, and third fluid compartments 211, 241, 271 may extend radially outward to the first, second, and third perimeter seams 223, 253, 283, respectively.

Each of the first, second, and third fabric wraps 220, 250, 280 may include one or more pad attachment mechanisms that are configured to secure the pads 210, 240, 270 together in the assembled state. For example, the first fabric wrap 220 includes a number (e.g., 1, 2, or more) first pad attachment mechanisms 231A configured to couple with a corresponding number of second pad attachment mechanism 261 of the second fabric wrap 250. Similarly, the first fabric wrap 220 includes a number (e.g., 1, 2, or more) fourth pad attachment mechanism 231A configured to couple with a corresponding number of third pad attachment mechanism 291 of the third fabric wrap 250. As such, in the assembled state, the first pad 210 is coupled with the both second pad 240 and the third pad 270 in a ready-to-use state prior to placement of the pad assembly 120 on the patient. In some instances of use, the second pad 240 and other the third pad 270 may omitted (i.e., separated from) from the pad assembly 120.

Each of the pad attachment mechanisms may be disposed along the respective perimeter seam of the fabric wrap and may extend outward from the respective perimeter seam and/or inward from the respective perimeter seam. The pad attachment mechanisms may include any suitable mechanism for attaching one pad to the other pad (e.g., the second pad 240 to the first pad 210) and detaching the one pad from the other pad, such as straps, bands, snaps, buttons, hook and loop systems (e.g., Velcro®) or adhesives, for example. In some embodiments, a pad attachment mechanism may simply include a portion of the patient contact panel or the outer panel. For example, a pad attachment mechanism of the one pad may include an adhesive configured to adhere directly to the patient contact panel or the outer panel of the other pad.

Each of the first, second, and third fabric wraps 220, 250, 280 may also include one or more patient attachment mechanisms that are configured to secure the pads 210, 240, 270 to the patient 50. The pad assembly 120 may include all or any subset of the patient attachment mechanisms described below. For example, in the illustrated embodiment, the first fabric wrap 220 may include a first shoulder patient attachment mechanism 232A configured to attach to a second shoulder patient attachment mechanism 232B for the purpose of securing the first pad 210 to the right shoulder area of the patient 50. The first fabric wrap 220 may further include a third shoulder patient attachment mechanism 233A configured to attach to a fourth shoulder patient attachment mechanism 233B for the purpose of securing the first pad 210 to the left shoulder area of the patient 50. The first fabric wrap 220 may further a number (e.g., 1, 2, or more) more first waist patient attachment mechanisms 234A configured to attach to a corresponding number of second waist patient attachment mechanisms 234B for the purpose of securing the first pad 210 to around the abdomen area of the patient 50.

The second fabric wrap 250 may include a number (e.g., 1, 2, or more) of first hip patient attachment mechanisms 262 configured to attach to a corresponding number of second hip patient attachment mechanisms 292 of the third fabric wrap 280 for the purpose of securing the second pad 240 and the third pad 270 to around the hip area of the patient 50. The second fabric wrap 250 may further include a number (e.g., 1, 2, or more) first thigh patient attachment mechanisms 263A configured to attach to a corresponding number of second thigh patient attachment mechanisms 263B for the purpose of securing the second pad 240 to the right thigh of the patient 50. In a similar fashion, the third fabric wrap 280 may further include a number (e.g., 1, 2, or more) of third thigh patient attachment mechanisms 293A configured to attach to a corresponding number of fourth thigh patient attachment mechanisms 293B for the purpose of securing the third pad 270 to the left thigh of the patient 50. Similar to the pad attachment mechanisms, the patient attachment mechanisms may include any suitable mechanism for attaching one portion of a pad to another portion of the pad or for attaching one pad to another pad (e.g., the second pad 240 to the third pad 270) and detaching the one portion from the other portion or the one pad from the other pad, such as straps, bands, snaps, buttons, hook and loop systems (e.g., Velcro®) or adhesives, for example. In some embodiments, a patient attachment mechanism may simply include portion of the patient contact panel or the outer panel.

FIG. 2C is a front view of the pad assembly 120 in the assembled state according to some embodiments disclosed herein. The first pad 210 is coupled with the second pad 240 and the third pad 270. The first fluid compartment 211 is fluidly coupled with the second fluid compartment 241 and the third fluid compartment 271. The second delivery connector 214 is coupled with the second inlet connector 242 enabling the TTM fluid to flow from the first fluid compartment 211 to the second fluid compartment 241. The second return connector 215 is coupled with the second outlet connector 243 enabling the TTM fluid to flow from the second fluid compartment 241 back to the first fluid compartment 211. The third delivery connector 216 is coupled with the third inlet connector 272 enabling the TTM fluid to flow from the first fluid compartment 211 to the third fluid compartment 271. The third return connector 217 is coupled with the third outlet connector 273 enabling the TTM fluid to flow from the third fluid compartment 271 back to the first fluid compartment 211.

The first fabric wrap 220 is attached to the second fabric wrap 250 via the number of first pad attachment mechanisms 231A coupled with the corresponding number of second pad attachment mechanisms 261. The first fabric wrap 220 is attached to the third fabric wrap 250 via the number of fourth pad attachment mechanisms 231B coupled with third pad the corresponding number of attachment mechanisms 291.

FIG. 2D illustrates a front view of the patient 50 having the pad assembly 120 placed thereon according to some embodiments. The first pad 210 is disposed across the back side (not shown) of the patient 50 with portions of the first pad 210 extending around the torso of the patient to the front side of the patent 50 so as to extend across the chest area and abdominal area of the patient. The first shoulder patient attachment mechanism 232A is attach to the second shoulder patient attachment mechanism 232B to secure the first pad 210 to the right shoulder area of the patient 50. The third shoulder patient attachment mechanism 233A is attach to the fourth shoulder patient attachment mechanism 233B to secure the first pad 210 to the left shoulder area of the patient 50. The number of first waist patient attachment mechanisms 234A are attached to the corresponding number of second waist patient attachment mechanisms 234B for the purpose to securing the first pad 210 around the abdomen area of the patient 50.

The number of first hip patient attachment mechanisms 262 are attached to the corresponding number of second hip patient attachment mechanisms 292 to secure the second pad 240 and the third pad 270 around the hip area of the patient 50. The number of first thigh patient attachment mechanisms 263A are attached to the corresponding number of second thigh patient attachment mechanisms 263B to secure the second pad 240 around the right thigh of the patient 50. The number of third thigh patient attachment mechanisms 293A are attached to the corresponding number of fourth thigh patient attachment mechanisms 293B to secure the third pad 270 around the left thigh of the patient 50.

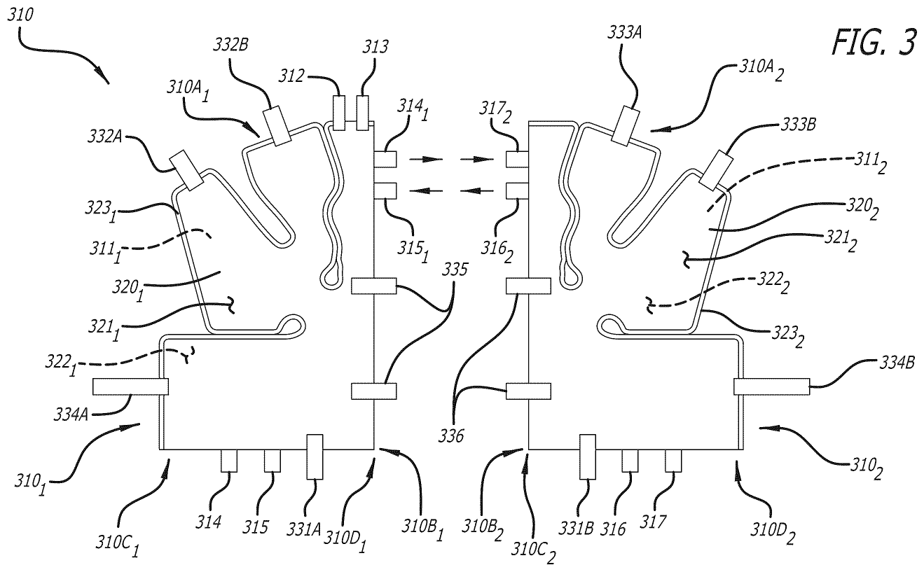

FIG. 3 is a front view of another embodiment of a first pad 310 that can, in certain respects, resemble components of the first pad 210 described in connection with FIGS. 2A-2D. It will be appreciated that all the illustrated embodiments may have analogous features. Accordingly, like features are designated with like reference numerals with the leading digit incremented to “3.” For example, the first top side of the first pad 210 is designated as 210A and the first top side of the first pad 310 is designated as 310A. Relevant disclosure set forth above regarding similarly identified features thus may not be repeated hereafter. Moreover, specific features of the first pad 210 and related components shown in FIGS. 2A-2D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the first pad 310 of FIG. 3. Any suitable combination of the features, and variations of the same, described with respect to the first pad 210 and components illustrated in FIGS. 2A-2D can be employed with the first pad 310 and components of FIG. 3, and vice versa. This pattern of disclosure applies equally to further embodiments depicted in subsequent figures and described hereafter.

The first pad 310 is divided into two sub-pads, namely a left first pad 310₁ and a right first pad 310₂. The left first pad 310₁ and the right first pad 310₂ are configured to selectively attach to each other and fluidly couple with one another. FIG. 3 illustrates the left first pad 310₁ and a right first pad 310₂ in a decoupled state. When the left first pad 310₁ and the right first pad 310₂ are the coupled together, the left first pad 310₁ and the right first pad 310₂ define the first pad 310 and resemble the features and functionality of the first pad 210. During use, either one of the left first pad 310₁ or the right first pad 310₂ may be decoupled from the pad assembly 120 so that the respective either one of the left first pad 310₁ or the right first pad 310₂ may be replaced. For illustration purposes, the left first pad 310₁ defines a top side 310A₁, a bottom side 310B₁, a left side 310C₁ and a right side 310D₁. In a similar fashion, the right first pad 310₂ defines a top side 310A₂, a bottom side 310B₂, a left side 310C₂ and a right side 310D₂. In a coupled state of the first pad 310, the right side 310D₁ of the left first pad 310₁ is disposed adjacent the left side 310C₂ of the right first pad 310₂.

The left first pad 310₁ includes a left first fluid compartment 311₁ having the second delivery connector 314 and the second return connector 315 fluidly coupled therewith. Similarly, the right first pad 310₂ includes a right first fluid compartment 311₂ having the third delivery connector 316 and the third return connector 317 coupled therewith. In the illustrated embodiment, the left first fluid compartment 311₁ may include the first inlet connector 312 and a first outlet connector 313. In other embodiments, the right first fluid compartment 311₂ may include the first inlet connector 312 and a first outlet connector 313.

The left first fluid compartment 311₁ is fluidly coupled with the right first fluid compartment 311₂ such that the TTM fluid is transferred from the left first fluid compartment 311₁ to the right first fluid compartment 311₂ and the TTM fluid is retuned from the right first fluid compartment 311₂ to the left first fluid compartment 311₁. The left first fluid compartment 311₁ includes a fourth delivery connector 314₁ configured to couple with a fourth inlet connector 317₂ to enable the transfer of the TTM fluid from the left first fluid compartment 311₁ to the right first fluid compartment 311₂. The left first fluid compartment 311₁ further includes a fourth return connector 315₁ configured to couple with a fourth outlet connector 316₂ to enable the return of the TTM fluid from the right first fluid compartment 311₂ to the left first fluid compartment 311₁. The fourth delivery connector 314₁ and the fourth return connector 315₁ may be disposed along the right side 310D₁ of the left first pad 310₁, and the fourth inlet connector 317₂ and the fourth outlet connector 316₂ may be disposed along the left side 310C₂ of the right first pad 310₂, although other arrangements are considered. Each of the fourth delivery connector 314₁, the fourth inlet connector 317₂, the fourth return connector 315₁, and the fourth outlet connector 316₂ includes a valve.

Each of the left first pad 310₁ and the right first pad 310₂ includes a fabric wrap similar to the first, second, and third fabric wraps 220, 250, 280. The left first pad 310₁ includes the left first fabric wrap 320₁ having the first patient contact panel 321₁ extending across the front side of the left first pad 310₁ and the first outer panel 322₁ extending across the back side of the left first pad 310₁, and the first perimeter seam 323₁. The left first fabric wrap 320₁ includes the number of pad attachment mechanisms 331A, the first shoulder patient attachment mechanism 332A configured to attach to the second shoulder patient attachment mechanism 332B, and the number of first waist patient attachment mechanisms 334A. Similarly, the right first pad 310₁ includes the right first fabric wrap 320₂ having the first patient contact panel 321₂ extending across the front side of the right first pad 310₂ and the first outer panel 322₂ extending across the back side of the right first pad 310₂, and the first perimeter seam 323₂. The right first fabric wrap 320₁ includes the number of first pad attachment mechanisms 331B, the first shoulder patient attachment mechanism 333A configured to attach to the second shoulder patient attachment mechanism 333B, and the number of first waist patient attachment mechanisms 334B.

The left first fabric wrap 320₁ is configured for selective attachment to the right first fabric wrap 320₂ to enable selective attachment of the left first pad 310₁ to the right first pad 310₂. A number (e.g., 1, 2 or more) left first pad attachment mechanisms 335 of the left first fabric wrap 320₁ are configured for selective attachment to corresponding number of right first pad attachment mechanisms 336 of the right first fabric wrap 320₁. The left first pad attachment mechanisms 335 may be disposed along the right side 310D₁ of the left first pad 310₁ and the right first pad attachment mechanisms 336 may be disposed along the left side 310C₂ of the right first pad 310₂.

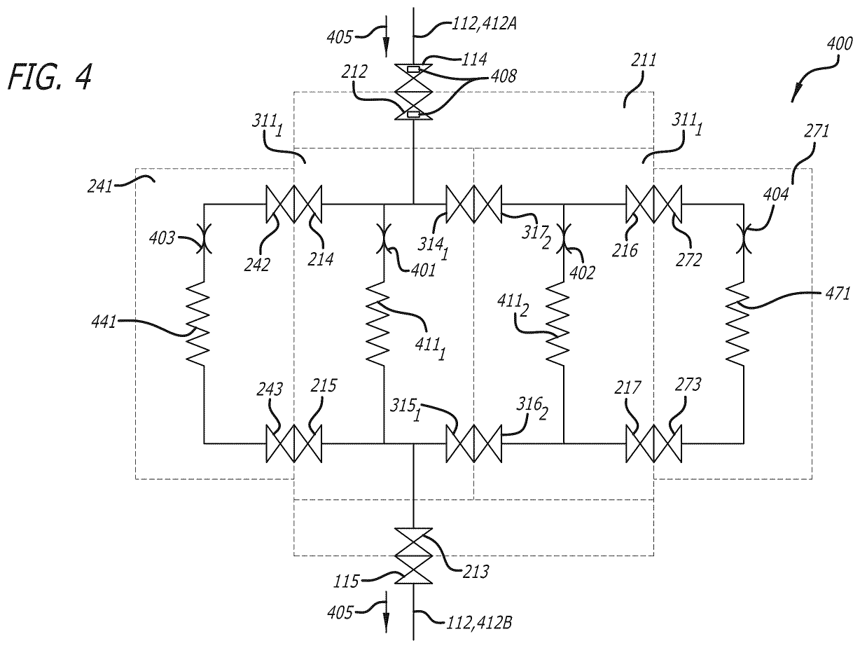

FIG. 4 is a fluid schematic diagram 400 of the pad assembly 120 in accordance with some embodiments disclosed herein. As shown, the TTM fluid 405 (illustrated in FIG. 4 as arrows which may or may not include the reference #405) flows from the TTM module 110 toward to the pad assembly 120 along a delivery pathway 412A of the FDL 112. The TTM fluid 405 enters the pad assembly 120 by passing through the first delivery connector 114 and the first inlet connector 212. The TTM fluid 405 exits the pad assembly 120 by passing through the first outlet connector 213 and the first return connector 115 before flowing along a fluid return pathway 412B of the FDL 112 toward the TTM module 110. The TTM fluid 405 flows through a circulation circuit of each of the first, second, and third fluid compartments 211, 241, 271. As shown in FIG. 4, in some embodiments, the left first fluid compartment 311₁ may be combined with the right first fluid compartment 311₂ to define the first fluid compartment 211.

Each of the fluid compartments includes a circulation circuit which defines the thermal energy transfer between the patient and the TTM fluid 405 circulating through the respective fluid compartment. The left first fluid compartment 311₁ includes the left first circulation circuit 411₁, the right first fluid compartment 311₂ includes the right first circulation circuit 411₂, the second fluid compartment 241 includes the second circulation circuit 441 and the third fluid compartment 271 includes the third circulation circuit 471. In the illustrated embodiment, the left first circulation circuit 411₁, the right first circulation circuit 411₂, the second circulation circuit 441, and the third circulation circuit 471 are disposed in a parallel arrangement with each other. However, in other embodiments, the second circulation circuit 441 may be disposed (either partially or fully) in a series arrangement with the left first circulation circuit 411₁, i.e., the TTM fluid 405 circulating through the second circulation circuit 441 may first circulate through all or a portion of the left first circulation circuit 411₁. Similarly, in other embodiments, the third circulation circuit 471 may be disposed (either partially or fully) in a series arrangement with the right first circulation circuit 411₂. Further still, the right first circulation circuit 411₂ may be disposed (either partially or fully) in a series arrangement with the left first circulation circuit 411₁.

The TTM fluid 405 flows from the left first fluid compartment 311₁ to the second fluid compartment 241 by passing through the second delivery connector 214 and the second inlet connector 242, and the TTM fluid 405 flows from the second fluid compartment 241 back to the left first fluid compartment 311₁ by passing through the second outlet connector 243 and the second return connector 215. Similarly, the TTM fluid 405 flows from the right first fluid compartment 311₂ to the third fluid compartment 271 by passing through the second third connector 216 and the third inlet connector 272, and the TTM fluid 405 flows from the third fluid compartment 271 back to the right first fluid compartment 311₂ by passing through the third outlet connector 273 and the third return connector 217.

As shown in FIG. 4, each of the first delivery connector 114 and the first inlet connector 212 may optionally include a valve 408. The valve 408 is configured to (i) allow TTM fluid 405 to flow through the first delivery connector 114 and the first inlet connector 212 when the first delivery connector 114 is coupled with the first inlet connector 212, and (ii) prevent the TTM fluid 405 from flowing through the first delivery connector 114 and the first inlet connector 212 when the first delivery connector 114 and the first inlet connector 212 are decoupled from each other. Although not shown, each of the first return connector 115, the first outlet connector 213, the second delivery connector 214, the second inlet connector 242, the second return connector 215, the second outlet connector 243, the third delivery connector 216, the third inlet connector 272, the third return connector 217, the third outlet connector 273, the fourth delivery connector 314₁, the fourth inlet connector 317₂, the fourth return connector 315₁, and the fourth outlet connector 316₂ may also optionally include the valve 408 such that TTM fluid 405 is allowed to flow through the respective connectors when coupled together and prevent TTM fluid 405 from flowing through the respective connectors when decoupled from each other. The valve 408 may advantageously prevent leaking of the TTM fluid 450 from the respective connector upon decoupling of the respective connector. Similarly, the valve 408 may advantageously prevent air ingress through the respective connector upon decoupling the respective connector.

In other words, whenever any of the connectors are connected to a corresponding connector, the valve 408 of the respective connector is open, and whenever any of the connectors are not connected to the corresponding connector, the valve 408 of the respective connector is closed. As such, the TTM fluid 405 is prevented from leaking from any connector when that connector is disconnected. Similarly, air is prevented from passing through the connector when that connector is disconnected.

As the left first circulation circuit 411₁, the right first circulation circuit 411₂, the second circulation circuit 441, and the third circulation circuit 471 may be disposed partially or entirely in a parallel arrangement with each other, the flow rate of the TTM fluid 405 through each of the left first circulation circuit 411₁, the right first circulation circuit 411₂, the second circulation circuit 441, and the third circulation circuit 471 may in some embodiments be different, i.e., not equal. Also, in some embodiments, the sizes of the fluid compartments (in terms of patient contact area and/or fluid volume) may be different (non-equal) and as such, it may be advantageous to regulate the portion of the TTM fluid 405 flow through the fluid compartments in accordance with the size of the compartments, e.g., a larger pad may warrant a greater flow rate. Similarly, pads of equal size may warrant equal flow rates of the TTM fluid 405. By way of one example, the size of the second fluid compartment 241 may be greater than the size of the left first fluid compartment 311₁. As such, it may be desirable to regulate the flow rate of the TTM fluid 405 such that a greater portion of the flow rate passes through the second fluid compartment 241 than passes through the left first fluid compartment 311₁. Such regulation may add balance to the thermal energy transfer defined by the left first fluid compartment 311₁ and the second fluid compartment 241. By way of a second example, the first fluid compartment 211 (i.e., the left first fluid compartment 311₁ combined with the right first fluid compartment 311₂) may be greater than the second fluid compartment 241 or the second fluid compartment 271. As such, it may be desirable to regulate the flow rate of the TTM fluid 405 such that a greater portion of the flow rate passes through the first fluid compartment 211 than passes through either of the second and third fluid compartments 241, 271.

In accordance with the forgoing, the pad assembly 120 may include a number of fluid restrictors to regulate the flow rate of the TTM fluid 405 through different fluid compartments, e.g., control a flow rate of the TTM fluid 405 within the fluid compartment in accordance with a desired flow rate or portion of the total flow rate of the TTM fluid 405. In the illustrated embodiment, the pad assembly 120 may include one or more of a left first restrictor 401 disposed in line with the left circulation circuit 411₁, a right first restrictor 402 disposed in line with the right circulation circuit 411_{2,} a second restrictor 403 in line with the second circulation circuit 441, or a third restrictor 404 in line with the third circulation circuit 471. The restrictors 401-404 may take any form suitable to restrict the flow rate of TTM fluid through the restrictor, such as a flow orifice or a flow regulator having a deflectable member, for example. In some embodiments, any of the restrictors 401-404 may be integral to the respective circulation circuit, integral to one of the connectors in line with the circulation circuit, integral to the valve 408, a separate component disposed in line with the circulation circuit, or any combination thereof.

In some embodiments, the left first restrictor 401 may be greater (i.e., more restrictive) than the second restrictor 403 so that a greater portion of the TTM flow rate passes through the second circulation circuit 441 than passes through the left first circulation circuit 411₁. Similarly, in some embodiments, the right first restrictor 402 may be greater (i.e., more restrictive) than the third restrictor 404 so that a greater portion of the TTM flow rate passes through the third circulation circuit 471 than passes through the right first circulation circuit 411₂. In some embodiments, either or both of the second restrictor 403 or the third restrictor 404 may be greater than the left first restrictor 401 in combination (i.e., parallel combination) with the right first restrictor 402 so that a greater portion of the TTM flow rate passes through the left first circulation circuit 411₁ combined with the right first circulation circuit 411₂ (i.e., the first fluid compartment 211). In some embodiments, any of the restrictors 401-404 may be omitted.

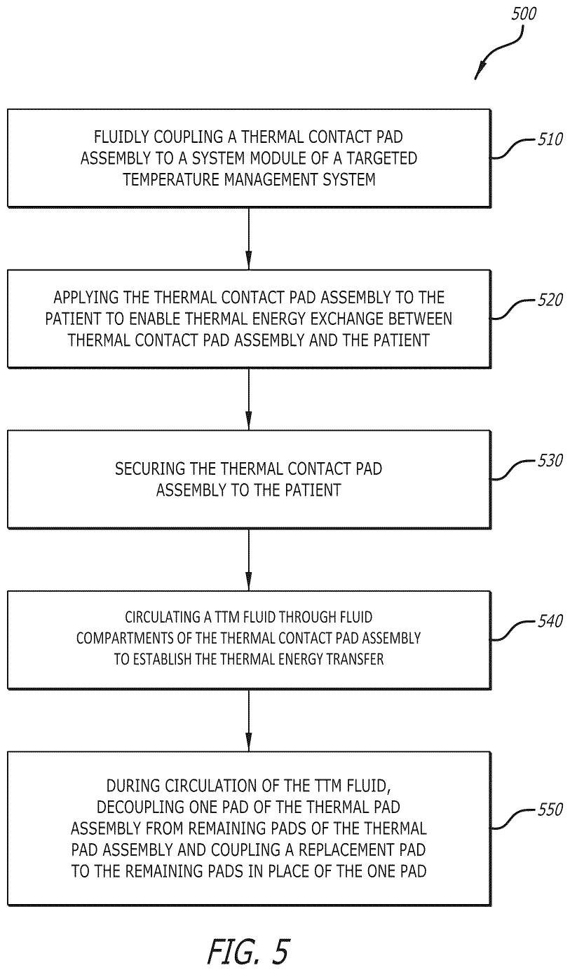

FIG. 5 illustrates a block diagram of a method 500 of exchanging thermal energy with a patient according to some embodiments disclosed herein. The method 500 may include all or any subset of the following steps, actions, procedures, or operations. The method 500 may include fluidly coupling a thermal contact pad assembly (pad assembly) to a system module of a targeted temperature management (TTM) system (block 510), that the system module may define the circulation of the TTM fluid through the pad assembly. In accordance with the method 500, the thermal contact pad assembly includes a plurality of thermal contact pads (pads), where each pad includes (i) a fluid compartment configured for circulation of a targeted TTM fluid therein to define the thermal energy exchange between the TTM fluid and the patient. Each pad may further include a fabric wrap defining a covering for the fluid compartment. According to such embodiments of the method 500, the fabric wrap includes (i) an outer panel, (ii) patient contact panel attached to the outer panel along a perimeter of the fabric wrap, and (iii) one or more attachment mechanisms configured to the pad to an adjacent one of the plurality of pads. The method 500 further includes applying the thermal contact pad assembly to the patient to enable thermal energy exchange between thermal contact pad assembly and the patient (block 520), so that each pad is thermally coupled with the patient. The method 500 may further include securing the thermal contact pad assembly to the patient (block 530). Securing the pad assembly may include securing each pad to the patent via the patient attachment mechanisms. The method 500 further includes, circulating the TTM fluid through fluid compartments of the thermal contact pad assembly to establish the thermal energy transfer (block 540). In some embodiments of the method 500, circulating the TTM fluid includes drawing (i.e., pulling) the TTM fluid through the fluid compartments such that a pressure within the fluid compartments is negative (i.e., below atmospheric pressure).

The method 500 may further include, during circulation of the TTM fluid, (i) decoupling one pad of the thermal pad assembly from remaining pads of the thermal pad assembly and (ii) coupling a replacement pad to the remaining pads in place of the one pad (block 550). In some embodiments, the decoupling of the one pad and the coupling a replacement pad is performed while the TTM fluid is continually circulating through the remaining pads.

In some embodiments of the method 500, decoupling the one pad from the remaining pads includes decoupling the attachment mechanisms of the fabric wrap of the one pad from the attachment mechanisms of the fabric wrap of at least one of the remaining pads. Similarly, coupling the replacement pad to the remaining pads includes coupling the attachment mechanisms of the fabric wrap of the replacement pad to the one or more attachment mechanisms of the fabric wrap of the at least one of the remaining pads.

In some embodiments of the method 500, decoupling the one pad from the remaining pads includes (i) decoupling a delivery connector of the remaining pads from an inlet connector of the one pad, and (ii) decoupling a return connector of the remaining pads from an outlet connector of the one pad. Similarly, coupling the replacement pad in place of the first pad includes (i) coupling the delivery connector of the remaining pads to an inlet connector of the replacement pad and (ii) coupling the return connector of the remaining pads to an outlet connector of the replacement pad.

In some embodiments of the method 500, the delivery connector and/or the inlet connector may include a valve in line therewith, where the valve is configured to (i) allow fluid flow through the respective connector when the delivery connector is coupled with the inlet connector, and (ii) prevent fluid flow through the respective connector when the delivery connector is decoupled coupled with the inlet connector. Similarly, in some embodiments of the method 500, the return connector and/or the outlet connector may include the valve in line therewith.

In some embodiments of the method 500, the first pad of the pad assembly may include a first sub-pad coupled with a second sub-pad of the first pad. As such, the method 500 may include decoupling the first sub-pad from the second sub-pad and coupling a replacement first sub-pad with the second sub-pad.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A thermal contact pad assembly configured for use in conjunction with a targeted temperature management (TTM) system, comprising:
a plurality of thermal contact pads (pads), each pad including:
a fluid compartment configured for circulation of a targeted TTM fluid therein to define a thermal energy exchange between the TTM fluid and a patient;
one of a plurality of inlet connectors in fluid communication with the fluid compartment, wherein each of the plurality the inlet connectors is configured to couple with any one of a plurality of corresponding fluid delivery connectors;
one of a plurality of outlet connectors in fluid communication with the fluid compartment, wherein each of the plurality the outlet connectors is configured to couple with any one of a plurality of a corresponding fluid return connectors,
wherein each of the delivery and return connectors is included by one of the pads or a fluid delivery line of the TTM system; and
a fabric wrap defining a covering for the fluid compartment, the fabric wrap including:
an outer panel; and
patient contact panel attached to the outer panel along a perimeter of the fabric wrap, the fluid compartment disposed between the outer panel and the patient contact panel; and
one or more pad attachment mechanisms configured to attach the pad to another one of the pads.

2. The pad assembly according to claim 1, wherein the one or more pad attachment mechanisms are configured for selective:
attachment of the pad to the other one of the pads, and
detachment of the pad from the other one of the pads.

3. The pad assembly according to claim 1, further including one or more patient attachment mechanisms configured to secure the pad assembly to the patient.

4. The pad assembly according to claim 3, wherein the patient attachment mechanisms are configured to secure one portion of the pad to another portion of the pad.

5. The pad assembly according to claim 1, wherein:
each inlet connector includes a first valve disposed in line therewith, the first valve configured to:
transition from a closed state to an open state upon coupling of the inlet connector with the corresponding fluid delivery connector, and
transition from the open state to the closed state upon decoupling of the inlet connector from the corresponding fluid delivery connector, and
each outlet connector includes a second valve disposed in line therewith, the second valve configured to:
transition from a closed state to an open state upon coupling of the outlet connector with the corresponding fluid return connector, and
transition from the open state to the closed state upon decoupling of the outlet connector from the corresponding fluid return connector.

6. The pad assembly according to claim 5, wherein:
each corresponding fluid delivery connector includes a third valve disposed in line therewith, the third valve configured to:
transition from a closed state to an open state upon coupling of the corresponding fluid delivery connector with the each inlet connector, and
transition from the open state to the closed state upon decoupling of the corresponding fluid delivery connector from the each inlet connector, and
each corresponding fluid return connector outlet connector includes a fourth valve disposed in line therewith, the fourth valve configured to:
transition from a closed state to an open state upon coupling of the corresponding fluid return connector with the each outlet connector, and
transition from the open state to the closed state upon decoupling of the corresponding fluid return connector from the each outlet connector.

7. The pad assembly according to claim 1, wherein each of the pads is configured for use individually in conjunction with the TTM system in the absence of the other pads of the pad assembly.

8. The pad assembly according to claim 7, wherein each pad is configured for fluidly individual coupling with the FDL.

9. The pad assembly according to claim 1, wherein one or more of the pads includes one or more of the corresponding fluid delivery connectors and one or more of the corresponding fluid return connectors in fluid communication with its respective fluid compartment.

10. The pad assembly according to claim 1, wherein one or more of the pads includes a fluid restrictor disposed in line with a circulation circuit of its respective fluid compartment, the fluid restrictor configured to control a flow rate of the TTM fluid within the fluid compartment.

11. The pad assembly according to claim 1, wherein:
the plurality of pads includes a first pad, a second pad, and a third pad, and
each of the first, second, and third pads defines:
a top side,
a bottom side disposed opposite the top side,
a left side, and
a right side disposed opposite the left side.

12. The pad assembly according to claim 11, wherein in an assembled state of the pad assembly:
the bottom side of the first pad is disposed adjacent the top side of the second pad and the top side of the third pad, and
the right side of the second pad is disposed adjacent the left side of the third pad.

13. The pad assembly according to claim 11, wherein in an assembled state of the pad assembly, the first pad is attached to both the second pad and the third pad via the one or more attachment mechanisms.

14. The pad assembly according to claim 11, wherein the inlet connector and the outlet connector of the first pad are coupled with a delivery connector and a return connector of the fluid delivery line, respectively.

15. The pad assembly according to claim 11, wherein the inlet connector and the outlet connector of the second pad are coupled with a first delivery connector and a first return connector of the first pad, respectively.

16. The pad assembly according to claim 11, wherein the inlet connector and the outlet connector of the third pad are coupled with a second delivery connector and a second return connector of the first pad, respectively.

17. The pad assembly according to claim 11, wherein:
the first pad is divided into a first sub-pad and a second sub pad such that the fluid compartment of the first pad is divided into a first sub-fluid compartment and a second sub-fluid compartment,
the first sub-pad includes the inlet connector and the outlet connector of the first pad in fluid communication with the first sub-fluid compartment,
the second sub-pad includes one of the plurality of inlet connectors and one of the plurality of outlet connectors in fluid communication with the second sub-fluid compartment, and
the first and second sub-pads include first and second sub-fabric wraps defining a covering therefor, each of the first and second sub-fabric wraps including:
an individual outer panel;
an individual patient contact panel attached to the individual outer panel along a perimeter of the corresponding sub-fabric wrap; and
a subset of the one or more attachment mechanisms, the subset configured to attach first sub-pad to the second sub-pad.

18. A method of exchanging thermal energy with a patient, comprising:
fluidly coupling a thermal contact pad assembly to a system module of a targeted temperature management (TTM) system, the thermal contact pad assembly, comprising:
a plurality of thermal contact pads (pads), each pad including:
a fluid compartment configured for circulation of a targeted TTM fluid therein to define a thermal energy exchange between the TTM fluid and a patient; and
a fabric wrap defining a covering for the fluid compartment, the fabric wrap including:
an outer panel;
patient contact panel attached to the outer panel along a perimeter of the fabric wrap; and
one or more attachment mechanisms configured to attach each pad to an adjacent one of the pads;
applying the thermal contact pad assembly to the patient;
securing the thermal contact pad assembly to the patient via one or more patient attachment mechanisms of the thermal contact pad assembly; and
circulating a TTM fluid through the fluid compartments to establish the thermal energy transfer.

19. The method of claim 18, further comprising:
decoupling one pad of the thermal pad assembly from remaining pads of the thermal pad assembly; and
coupling a replacement pad to the remaining pads in place of the one pad.

20. The method of claim 19, wherein:
decoupling the one pad from the remaining pads includes decoupling one or more attachment mechanisms of the fabric wrap of the one pad from one or more attachment mechanisms of the fabric wrap at least one of the remaining pads, and
coupling the replacement pad to the remaining pads includes coupling one or more attachment mechanisms of the fabric wrap of the replacement pad to the one or more attachment mechanisms of the fabric wrap of the at least one of the remaining pads.

21. The method of claim 19, further comprising maintaining circulation of the TTM fluid within the fluid compartments of the remaining pads during the decoupling of the one pad and the coupling of the replacement pad.

22. The method of claim 19, wherein:
decoupling the one pad from the remaining pads includes:
decoupling a delivery connector of the remaining pads from an inlet connector of the one pad; and
decoupling a return connector of the remaining pads from an outlet connector of the one pad, and
coupling the replacement pad in place of the first pad includes:
coupling the delivery connector of the remaining pads to an inlet connector of the replacement pad; and
coupling the return connector of the remaining pads to an outlet connector of the replacement pad.
